(19) United States
(12) Patent Application Publication
(10) Pub. No.: US 2026/0364038 A1
(43) Pub. Date: Nov. 19, 2026

(54) VASODILATOR, NITRIC OXIDE SYNTHASE PRODUCTION PROMOTER, AND USE OF SAME

(71) Applicant: J-OIL MILLS, INC., Tokyo (JP)

(72) Inventors: Rina HIROOKA, Tokyo (JP); Masayoshi SAKAINO, Tokyo (JP); Saki NISHIMURA, Tokyo (JP); Ryosuke AOKI, Tokyo (JP)

(21) Appl. No.: 19/130,853

(22) PCT Filed: Dec. 4, 2023
(86) PCT No.: PCT/JP2023/043272

§ 371 Date: May 16, 2025

(30) Foreign Application Priority Data
Dec. 12, 2022 (JP) 2022-198072

(51) Int. Cl. A61K 31/20

(52) U.S. Cl. CPC A61K 31/20

(57) ABSTRACT

There are provided, as novel materials having an exceptional vasodilation action: a vasodilator containing, as an active ingredient, at least one selected from the group consisting of C13 or higher and C15 or lower fatty acids, and fatty acid esters that contain said fatty acids; and a nitric oxide synthase production promoter containing, as an active ingredient, at least one selected from the group consisting of C13 or higher and C15 or lower fatty acids, and fatty acid esters that contain said fatty acids.

### TECHNICAL FIELD

The present invention relates to a vasodilator, a nitric oxide synthase production promoter, and a use of the same.

### BACKGROUND ART

In order to improve the quality of life activities and extend healthy life expectancy, it is important to maintain vascular function. Vascular endothelial cells, which are present inside blood vessels, are known to produce nitric oxide (NO), which works to dilate blood vessels. When blood vessels are dilated, blood pressure decreases and blood flow circulation improves. Therefore, vasodilation can be anticipated to prevent or ameliorate various conditions such as hypertension and arteriosclerosis.

As technology relating to a vasodilation action, for example, Patent Document 1 discloses a vasodilator containing, as an active ingredient, at least one selected from the group consisting of citric acid and salts thereof. Patent Document 2 discloses an endothelial nitric oxide synthase production promoter and a vasodilator characterized by containing piperine.

### RELATED ART DOCUMENTS

### Patent Documents

Patent Document 1: Japanese Laid-open Patent Application No. 2020-186205
Patent Document 2: Japanese Laid-open Patent Application No. 2016-222588

### DISCLOSURE OF THE INVENTION

### Problems the Invention is Intended to Solve

Against this backdrop, it is still desirable to provide a novel material having an exceptional vasodilation action. Accordingly, it is an object of the present invention to provide a novel material having an exceptional vasodilation action.

### Means for Solving the Aforementioned Problems

The inventors carried out thorough investigations in order to achieve the aforementioned object and perfected the present invention. Specifically, according to the present invention, there are provided the below-described vasodilator, nitric oxide synthase production promoter, food or beverage composition, oil and fat composition, supplement, and medicinal composition.
[1] A vasodilator containing, as an active ingredient, at least one selected from the group consisting of C13 or higher and C15 or lower fatty acids, and fatty acid esters that contain said fatty acids.
[2] The vasodilator according to [1] above, wherein the fatty acid is a saturated fatty acid.
[3] The vasodilator according to [1] or [2] above, wherein the fatty acid ester is a glycerol fatty acid ester.
[4] A nitric oxide synthase production promoter containing, as an active ingredient, at least one selected from the group consisting of C13 or higher and C15 or lower fatty acids, and fatty acid esters that contain said fatty acids.
[5] A food or beverage composition containing the agent set forth in any one of [1] to [4].
[6] An oil and fat composition containing the agent set forth in any one of [1] to [4].
[7] A supplement containing the agent set forth in any one of [1] to [4].
[8] A medicinal composition containing the agent set forth in any one of [1] to [4].

### Effect of the Invention

According to the present invention, it is possible to provide a novel vasodilator and nitric oxide synthase production promoter.

### MODE FOR CARRYING OUT THE INVENTION

Embodiments of the present invention are described below. Hyphens in numeric value ranges indicate that a parameter is equal to or greater than one numeric value and equal to or less than another numeric value, and include the numeric values at both ends of the range, unless otherwise indicated. When upper-limit values and lower-limit values for numeric value ranges are indicated, the upper-limit values and lower-limit values can be combined as appropriate, and the resulting numeric value ranges are also disclosed.

### (Vasodilator and Nitric Oxide Synthase Production Promoter)

The vasodilator according to the present invention contains, as an active ingredient, at least one selected from the group consisting of C13 or higher and C15 or lower fatty acids, and fatty acid esters that contain said fatty acids.

The nitric oxide synthase production promoter according to the present invention contains, as an active ingredient, at least one selected from the group consisting of C13 or higher and C15 or lower fatty acids, and fatty acid esters that contain said fatty acids.

The nitric oxide synthase production promoter has an action for promoting production of endothelial nitric oxide synthase (eNOS), which is localized in vascular endothelial cells. Promoting the production of eNOS promotes production of nitric oxide in the vascular endothelium and relaxes vascular smooth muscles, resulting in blood vessels being dilated.

The vasodilator has an action for dilating blood vessels due to relaxation of the vascular smooth muscles caused by nitric oxide produced by the eNOS.

Due to having an action for dilating blood vessels as described above, the aforementioned vasodilator and nitric oxide synthase production promoter can reduce blood pressure and improve blood flow. Thus, the vasodilator and nitric oxide synthase production promoter can be suitably used in preventing or ameliorating vasoconstrictive conditions (which refers to conditions that appear as a result of blood vessels shrinking, contracting, becoming blocked, etc.) or in preventing or treating such conditions. Examples of vasoconstrictive conditions include hypertension, arteriosclerosis, angina, myocardial infarction, sensitivity to cold, stiff shoulders, constipation, headache, cerebral infarction, and cerebral hemorrhage.

### (Fatty Acid)

There is no particular limitation as to the source, etc., of the fatty acid that is an active ingredient in the present invention, provided that the fatty acid is a C13 or higher and C15 or lower fatty acid. For example, the fatty acid may be derived from a natural substance or may be synthetic. Additionally, it is preferable that the C13 or higher and C15 or lower fatty acid can be used upon being broken down within the body; for example, a fatty acid ester that contains said fatty acid may be employed. The fatty acid is preferably formulated from a natural substance in order to be provided in the form of a food or beverage composition or a medicinal composition. Hereinafter, for convenience of description, the C13 or higher and C15 or lower fatty acid used in the present invention may be referred to as “the fatty acid (C13-15),” the fatty acid ester that contains the C13 or higher and C15 or lower fatty acid may be referred to as “the fatty acid (C13-15) ester,” and at least one selected from the group consisting of the C13 or higher and C15 or lower fatty acid and the fatty acid ester that contains said fatty acid may be referred to as “the fatty acid (C13-15), etc.”

Examples of the fatty acid (C13-15) include tridecanoic acid (C13:0), tetradecanoic acid (C14:0), and pentadecanoic acid (C15:0). The fatty acid (C13-15) is not limited to these saturated fatty acids; it is permissible to use an unsaturated fatty acid having a double bond in part of the fatty acid alkyl chain, and it is also permissible for part of the fatty acid alkyl chain to be branched. The fatty acid (C13-15) is preferably a saturated fatty acid.

Examples of the fatty acid (C13-15) ester include glycerol fatty acid esters, sucrose fatty acid esters, organic-acid fatty acid esters, fatty acid alcohol esters, sphingolipids, sorbitan fatty acid esters, polypropylene fatty acid esters, and sterol esters that contain the fatty acid (C13-15). Among these fatty acid esters, glycerol fatty acid esters are preferred. Glycerol fatty acid esters are esters having glycerol as a constituent component; specific examples include triacyl glycerol, diacyl glycerol, monoacyl glycerol, glycerophospholipids, and glyceroglycolipids. Among these glycerol fatty acid esters, triacyl glycerol is preferred.

One type of fatty acid (C13-15) may be used alone, or a plurality of types thereof may be used in combination. In cases where an enantiomer or a diastereomer is present, a single-type variety may be used alone as a single-molecule species thereof, or a plurality of types of a plurality of molecular species of isomers thereof may be used in combination.

As a method for obtaining the fatty acid (C13-15), etc., from a natural substance, large amounts of triglyceride-containing lipids that contain fatty acids (C14) as constituent fatty acids are included in, e.g., coconut oil, palm kernel oil, milk fat, and other oils and fats, although the method is not limited thereto. Additionally, Japanese Laid-open Patent Application No. 2016-89025 makes it clear that large amounts of triglycerides that contain fatty acids (C13) and fatty acids (C15) as constituent fatty acids are produced by algae of the genus Aurantiochytrium, which are known as abundant-oil-and-fat-producing algae. Thus, fatty acids (C13-15) can be efficiently obtained by using such edible oils and fats or abundant-oil-and-fat-producing algae as raw materials. Typically, for example, n-hexane, chloroform, diethyl ether, methanol, ethanol, or another organic solvent, or an organic solvent mixture thereof, is added to, inter alia, a dried article of algae or another natural substance, and extraction is carried out, whereby triglyceride-containing lipids that contain the fatty acids (C13) and the fatty acids (C15) as constituent fatty acids are obtained. Polar lipids may be removed from the resulting triglyceride-containing lipids by adsorption using silica gel, acid clay, activated clay, an ion exchange resin, or the like to obtain triglycerides. Alternatively, impurities may be removed from triglyceride-containing lipids by means of a typical step for refining plant-based oils and fats, i.e., a degumming step, a deacidification step, a decoloration step, or a deodorization step, to obtain refined triglyceride-containing lipids. Furthermore, the resulting triglycerides, triglyceride-containing lipids, or refined triglyceride-containing lipids can be subjected to a sodium hydroxide treatment or a hydrolysis treatment performed using a lipase or the like to isolate the fatty acids from the glycerol ester structure. The isolated fatty acids may furthermore be separated or refined by means of a method such as molecular distillation, urea addition, or column chromatography. Additionally, the resulting fatty acids, triglycerides, triglyceride-containing lipids, or refined triglyceride-containing lipids may be employed as raw materials and used in transesterification or ester synthesis for the triglycerides.

When a natural substance is used to prepare an ingredient containing the fatty acid (C13-15), etc., for example, it is preferable to use an ingredient in which the amount of the fatty acid (C13-15), etc. (purity), is raised to 1 mass % or higher, more preferable to use an ingredient in which the amount is raised to 2 mass % or higher, even more preferable to use an ingredient in which the amount is raised to 5 mass or higher, and particularly preferable to use an ingredient in which the amount is raised to 10 mass % or higher. Alternatively, it is preferable to use an ingredient in which the total amount of fatty acids other than the fatty acid (C13-15) is 99 mass % or lower, more preferable to use an ingredient in which the total amount is 95 mass % or lower, even more preferable to use an ingredient in which the total amount is 90 mass % or lower and particularly preferable to use an ingredient in which the total amount is 80 mass % or lower.

The fatty acid (C13-15), etc., can be quantified using a method that is well known to persons skilled in the art. For example, the fatty acid (C13-15), etc., can be analyzed using gas chromatography, liquid chromatography, or the like, which are defined in “The JOCS Standard Methods for the Analysis of Fats, Oils and Related Materials,” and quantified by application to the concentration obtained when a discretionarily defined standard sample is analyzed. In the description of the present application, the fatty acid (C13-15), etc., can be quantitatively identified by being handled as the total amount of one or a plurality of types of fatty acids (C13-15), etc., that can be detected.

The vasodilator and the nitric oxide synthase production promoter according to the present invention may be composed of only the aforementioned active ingredient or, in accordance with the specific form of the vasodilator and the nitric oxide synthase production promoter, may contain other components permissible in foods and beverages or medicines in addition to the active ingredient.

The vasodilator and the nitric oxide synthase production promoter according to the present invention may be in the form of a solid, a liquid (including solutions and suspensions), a paste, or another suitable form.

There is no particular limitation as to the usage form of the vasodilator and the nitric oxide synthase production promoter according to the present invention, provided that the vasodilator and the nitric oxide synthase production promoter are used such that the fatty acid (C13-15), etc., is administered to a subject. For example, the fatty acid (C13-15), etc., may be incorporated into a prescribed dosage form and administered in a desired form as appropriate by means of oral administration, topical application, inhalation, or intramuscular injection. The vasodilator and the nitric oxide synthase production promoter may be administered once per day or may be divided and administered a plurality of times per day.

The vasodilator and the nitric oxide synthase production promoter according to the present invention are not limited to use in humans but rather can also be applied to non-human animals such as cows, pigs, chickens, sheep, horses, and other livestock, as well as dogs, cats, and other pets. The present invention is preferably used in humans, and more preferably is suitably used in middle-aged and elderly healthy persons (specifically healthy persons aged 40 or older).

There is no particular limitation as to the amount of the fatty acid (C13-15), etc., in the vasodilator and the nitric oxide synthase production promoter, provided that the amount is adjusted as appropriate from the standpoint of ensuring a desired administration amount. For example, the amount may be 0.1 mass % or higher, 0.2 mass % or higher, 0.5 mass % or higher, 1 mass % or higher, 2 mass % or higher, 5 mass % or higher, 10 mass % or higher, or 30 mass % or higher. Additionally, the amount may be 100 mass % or lower, 90 mass % or lower, 80 mass or lower, 60 mass % or lower, 50 mass % or lower, or 30 mass % or lower. The ingredient via which the fatty acid (C13-15), etc., is provided may constitute the vasodilator and the nitric oxide synthase production promoter.

It is generally known that enzymes that isolate free fatty acids from esters are present in the body, as is the case with lingual lipase present in the oral cavity, gastric lipase present in the stomach, pancreatic lipase secreted in pancreatic juice, and the like. Furthermore, triglycerides and other esters included in blood are broken down into free fatty acids and glycerol by means of lipoprotein lipase present on the surfaces of cells in muscle and the like. Moreover, lipases that isolate free fatty acids from glycerophospholipids, which are esters, by means of hydrolysis are included among phospholipases. Additionally, it is known that receptors that take in a variety of free fatty acids, such as GPR 40, GPR 41, GPR 43, and GPR120, which are G-protein-coupled receptors (GPRs), are present on cell surfaces. Thus, the fatty acid (C13-15) can be isolated from the fatty acid ester of the fatty acid (C13-15), which is administered to the subject, at a suitable time and as appropriate within the body of the subject, and the effect of the fatty acid can be exhibited within the body.

When the vasodilator and the nitric oxide synthase production promoter according to the present invention are to be orally ingested by humans, the administration amount of the fatty acid (C13-15), etc., can be determined as appropriate according to the age or health state of the person to whom the fatty acid is to be administered, an administration continuance period, the frequency of administration, or the like. As an example of typical administration amounts, the administration amount may be within the range of 5 mg or higher and 5,000 mg or lower, within the range of 10 mg or higher and 3,000 mg or lower, within the range of 50 mg or higher and 2,000 mg or lower, or within the range of 100 mg or higher and 1,000 mg or lower per day for a 60-kg adult.

### (Food or Beverage Composition)

The food or beverage composition according to the present invention contains at least one of the vasodilator and the nitric oxide synthase production promoter, and may, as needed, furthermore contain additives or the like that are ordinarily used in foods and beverages.

Examples of the food or beverage composition include oil and fat compositions, confectionery (e.g., potato chips and other snack foods, cookies or cakes and other baked confectionery, traditional Japanese confectionery, chocolate, candy, pudding, jelly, ice cream, gummies, gum, and the like), breads (e.g., sweet pastries, dressed breads, croissants, Danish pastries, and the like), noodles (e.g., ramen, udon, pasta, and the like), rice foods (e.g., rice balls, rice gruel, fried rice, and the like), cereal foods (e.g., corn flakes, oatmeal, and the like), dairy products (e.g., cheese, yogurt, and the like), processed meat products (e.g., ham, sausage, and the like), processed seafood products (e.g., kamaboko (*1), fish sausage, and the like), condiments (e.g., mayonnaise, sauces, dressings, and the like), soups (e.g., miso soup, vegetable soups, and the like), processed foods and beverages (e.g., boiled foods, deep-fried foods, grilled foods, curry, and the like), pre-mixed flours (e.g., okonomiyaki flour, kara-age flour (*2), mixed confectioners' flour, and the like), solid roux (e.g., curry roux and the like), beverages (e.g., beer and other alcoholic beverages, sports drinks, lactic acid beverages, vegetable juice and other soft drinks, black tea and other teas, coffee, and the like), elder-oriented foods and beverages (e.g., liquid foods and the like), health foods, health-promoting foods, designated health foods, function-labeled foods, functional nutritional foods, nutritional supplements, supplements, protein, and animal feeds.

The food or beverage composition according to the present invention contains at least one of the vasodilator and the nitric oxide synthase production promoter, and therefore can be used as a vasodilating food or beverage composition or a nitric-oxide-synthase-production-promoting food or beverage composition. Additionally, the food or beverage composition can be suitably used for preventing or ameliorating the vasoconstrictive conditions described above.

### (Oil and Fat Composition)

The oil and fat composition can specifically be obtained by blending a diluent, an auxiliary agent, an emulsifier, a pH adjuster, or the like with an edible oil and fat as necessary and can be made in a discretionary form, such as a liquid, a solid, a powder, or a paste, by means of a publicly known method. Examples of the form of the oil and fat composition include liquid oils and fats, margarines, fat spreads, shortening, and powdered oils and fats that are mainly composed of oil and fat components. Alternatively, the oil and fat composition may be prepared in the form of a solution, a powder, a gel, a granulated article, or the like that has a low blending amount of oil and fat components. Additionally, for example, in the case of a powdered oil and fat composition, corn syrup or another auxiliary agent can be used, and an emulsifier can furthermore be added to prepare an emulsified raw material that is subsequently powderized. Examples of the means for powderization include spray drying and freeze drying.

Examples of the edible oil and fat include: rapeseed oil (including high-oleic-acid rapeseed oil), soybean oil (including high-oleic-acid soybean oil), palm oil, palm kernel oil, corn oil, olive oil, sesame oil, safflower oil (including high-oleic-acid safflower oil), sunflower oil (including high-oleic-acid sunflower oil), cottonseed oil, rice bran oil, peanut oil, coconut oil, grapeseed oil, cacao butter, and other plant-based oils and fats; algae oils; beef tallow, pig lard, chicken fat, milk fat, fish oil, and other animal-based oils and fats; and medium-chain fatty acid triglycerides. Additional examples include fractionated oils (medium-melting-point fractionated palm oil, soft fractionated palm oil, hard fractionated palm oil, etc.), transesterified oils, hydrogenated oils, and other processed oils and fats obtained using the aforementioned edible oils and fats. One edible oil and fat may be used alone, or two or more edible oils and fats may be mixed together.

An adjuvant that is ordinarily added to edible oil and fat compositions may be blended with the oil and fat composition. Examples of the adjuvant include antioxidants, defoamers, emulsifiers, flavorings, flavor-imparting agents, colorants, and bioactive substances. Specific examples include lignan, coenzyme Q, γ-oryzanol, tocopherol, and silicone.

There is no particular limitation as to the amount of the fatty acid (C13-15), etc., that is incorporated into the oil and fat composition. For example, the amount may be 0.05 mass % or higher, 0.1 mass % or higher, 0.2 mass % or higher, 0.5 mass % or higher, 1 mass % or higher, 2 mass % or higher, 5 mass % or higher, 8 mass % or higher, 10 mass % or higher, or 12 mass or higher. Additionally, the amount may be 100 mass % or lower, 90 mass % or lower, 80 mass % or lower, 60 mass % or lower, 50 mass % or lower, 30 mass % or lower, 20 mass % or lower, 15 mass % or lower, or 10 mass % or lower.

### (Supplement)

Examples of the form of the supplement include tablets (including uncoated tablets, sugar-coated tablets, effervescent tablets, film-coated tablets, chewable tablets, lozenges, and the like), pills, capsules (including hard capsules, soft capsules, seamless capsules, and the like), granules, powders, liquids, syrups, jellies, candies, and gummies.

There is no particular limitation as to the amount of the fatty acid (C13-15), etc., that is incorporated into the supplement. For example, the amount may be 0.05 mass % or higher, 0.1 mass % or higher, 0.2 mass % or higher, 0.5 mass or higher, 1 mass % or higher, 2 mass % or higher, 5 mass % or higher, 8 mass % or higher, 10 mass % or higher, or 12 mass % or higher. Additionally, the amount may be 100 mass % or lower, 90 mass % or lower, 80 mass % or lower, 60 mass % or lower, 50 mass % or lower, 30 mass % or lower, 20 mass % or lower, 15 mass % or lower, or 10 mass % or lower.

### (Medicinal Composition)

The medicinal composition according to the present invention contains at least one of the vasodilator and the nitric oxide synthase production promoter, and may, as needed, furthermore contain additives or the like that are ordinarily used in medicinal compositions. The medicinal composition may be a medicine or a quasi-medicine.

Examples of the medicinal composition include: tablets (including uncoated tablets, sugar-coated tablets, effervescent tablets, film-coated tablets, chewable tablets, lozenges, and the like), pills, capsules (including hard capsules, soft capsules, seamless capsules, and the like), granules, powders, liquids, syrups, jellies, candies, gummies, and other orally administrated agents; injections, drips, suppositories, and other non-orally administered agents; and ointments, plasters, and other external-use agents.

The medicinal composition according to the present invention contains at least one of the vasodilator and the nitric oxide synthase production promoter, and therefore can be used as a vasodilating medicinal composition or a nitric-oxide-synthase-production-promoting medicinal composition. Additionally, the medicinal composition can be suitably used for preventing or ameliorating the vasoconstrictive conditions described above.

### Examples

The present invention is more specifically described via test examples below, but these test examples do not in any way limit the scope of the present invention.

The materials and methods used in the present test examples are as follows.

### [1. Cells]

HUVECs (made by Lonza; catalog no.: C2519AS)

### [2. Culture Medium]

EGM-2 (made by Lonza; catalog no.: CC-3162)

### [3. Preparation of Substances Under Test]

The substances under test in (1) to (3) below were each dissolved in dimethyl sulfoxide (DMSO) (made by Nacalai Tesque) to prepare solutions having a concentration of 30 mM, and the solutions were added to the culture medium at a 1/1000 proportion to prepare culture media in which the target final concentration of the respective substances under test was 30 μM (Examples 1 to 3).

Additionally, as a positive control, the substance under test in (4) below, which is known to have an action for promoting the production of endothelial nitric oxide synthase, was dissolved in DMSO to prepare a solution having a concentration of 1 mM, and the solution was added to the culture medium at a 1/1000 proportion to prepare a culture medium in which the target final concentration of the substance under test was 1 μM (Control example 2).

### (Substances Under Test)

(1) Tetradecanoic acid (C14:0) (purity: 100%; made by Sigma-Aldrich)
(2) Pentadecanoic acid (C15:0) (purity: 98.8%; made by Sigma-Aldrich)
(3) Tridecanoic acid (C13:0) (purity: 99.2%; made by Sigma-Aldrich)
(4) Fluvastatin (made by Tokyo Chemical Industry Co., Ltd.; model no.: F0820)

### [4. Treatment of Substances Under Test]

The HUVECs were thawed in a T75 flask and cultured in a CO₂ incubator (“SCA-165DRS” made by Astec Co., Ltd.; conditions: 37 °C, 5% CO₂, humid; same below) using the EGM-2. The cells were isolated using a 0.05% concentration trypsin solution (“Trypsin-EDTA (0.05%), phenol red” made by Thermo Fisher Scientific; catalog no.: 25300054), sown in a 96-well plate using the culture medium, and cultured in the CO₂ incubator. The next day, the culture medium was replaced with the culture media prepared in section “(3. Preparation of substances under test)” above, and the cells were then cultured for 24 hours in the CO₂ incubator to carry out treatment using the substances under test.

[5. RNA Extraction, qPCR]

After the treatment, a culture medium supernatant was recovered, RNA was extracted from the cells using a FastLane Cell Probe kit (made by QIAGEN; catalog no.: 215011), OneStep real-time RT-PCR was carried out using a Taqman probe, and the relative eNOS genetic expression amount was calculated by means of the ddCt method. The relative eNOS genetic expression amount was derived by dividing the eNOS genetic expression amount by the GAPDH genetic expression amount.

### [6. Results]

The resulting relative eNOS genetic expression amounts are shown in table 1. A significant difference between the substance-under-test treatment groups (control example 2 and the examples) and control example 1 was certified by means of Dunnett's test. The results are collectively shown in Table 1.

TABLE 1

| | | Control | Control | | | |
|---|---|---|---|---|---|---|
| | | example 1 | example 2 | Example 1 | Example 2 | Example 3 |
| Substance-under- | Substance under | None | Fluvastatin | Tetradecanoic | Pentadecanoic | Tridecanoic |
| test treatment | test | added | | acid (C14:0) | acid (C15:0) | acid (C13:0) |
| | Final | — | 1 | 30 | 30 | 30 |
| | concentration | | | | | |
| | (μM) | | | | | |
| Relative eNOS | n1 | 0.9 | 2.45 | 1.27 | 1.15 | 1.15 |
| genetic | n2 | 0.92 | 2.57 | 1.26 | 1.28 | 1.33 |
| expression amount | n3 | 1.01 | 2.57 | 1.31 | 1.2 | 1.26 |
| | n4 | 1.03 | 2.89 | 1.12 | 1.36 | 1.28 |
| | n5 | 1.14 | 2.61 | 1.33 | 1.3 | 1.24 |
| | Average value | 1.00 | 2.62 | 1.26 | 1.26 | 1.25 |
| | of n1-n5 | | | | | |
| | Standard error | 0.04 | 0.07 | 0.04 | 0.04 | 0.03 |
| | Significant | — | ** | ** | ** | ** |
| | difference* | | | | | |

*Level of significance across both sides: 5%.
** Level of significance across both sides: 1%.

As shown in table 1, the relative eNOS genetic expression amount in the examples was significantly higher than that in control example 1, similarly to the case with the fluvastatin in control example 2. Therefore, it was ascertained that tetradecanoic acid, pentadecanoic acid, and tridecanoic acid have an action for promoting production of endothelial nitric oxide synthase.

1. A method for vasodilator comprising, administering to a subject in need thereof, as an active ingredient, at least one selected from the group consisting of C13 or higher and C15 or lower fatty acids, and fatty acid esters that contain said fatty acids.

2. The method for vasodilator according to claim 1, wherein the fatty acid is a saturated fatty acid.

3. The method for vasodilator according to claim 1, wherein the fatty acid ester is a glycerol fatty acid ester.

4. A method for promotion of nitric oxide synthase production comprising, administering to a subject in need thereof, as an active ingredient, at least one selected from the group consisting of C13 or higher and C15 or lower fatty acids, and fatty acid esters that contain said fatty acids.

5. The method for vasodilator according to claim 1, wherein the active ingredient is contained in a form of a food or beverage composition.

6. The method for vasodilator according to claim 1, wherein the active ingredient is contained in a form of an oil and fat composition.

7. The method for vasodilator according to claim 1, wherein the active ingredient is contained in a form of a supplement.

8. The method for vasodilator according to claim 1, wherein the active ingredient is contained in a form of a medicinal composition.

9. The method for promotion of nitric oxide synthase production according to claim 4, wherein the fatty acid is a saturated fatty acid.

10. The method for promotion of nitric oxide synthase production according to claim 4, wherein the fatty acid ester is a glycerol fatty acid ester.

11. The method for promotion of nitric oxide synthase production according to claim 4, wherein the active ingredient is contained in a form of a food or beverage composition.

12. The method for promotion of nitric oxide synthase production according to claim 4, wherein the active ingredient is contained in a form of an oil and fat composition.

13. The method for promotion of nitric oxide synthase production according to claim 4, wherein the active ingredient is contained in a form of a supplement.

14. The method for promotion of nitric oxide synthase production according to claim 4, wherein the active ingredient is contained in a form of a medicinal composition.
